(19) United States
(12) Patent Application Publication
(10) Pub. No.: US 2022/0005582 A1
(43) Pub. Date: Jan. 6, 2022

(54) SYSTEM AND METHOD FOR PERSONALIZATION AND OPTIMIZATION OF DIGITAL PATHOLOGY ANALYSIS

(71) Applicant: IBEX MEDICAL ANALYTICS LTD., Tel Aviv (IL)

(72) Inventors: Chaim LINHART, Hod Hasharon (IL); Lilach BIEN, Rehovot (IL); Ronen HELED, Kiryat-Ono (IL); Joseph MOSSEL, Tel Aviv (IL)

(73) Assignee: IBEX MEDICAL ANALYTICS LTD., Tel Aviv (IL)

(21) Appl. No.: 17/283,805

(22) PCT Filed: Oct. 10, 2019
(86) PCT No.: PCT/IL2019/051109

§ 371 Date: Apr. 8, 2021

Related U.S. Application Data
(60) Provisional application No. 62/743,559, filed on Oct. 10, 2018.

(51) Int. Cl. G16H 30/20; G16H 10/40; G16H 50/20; G16H 50/30; G06T 7/11; G06T 7/00; G06T 3/40; G06T 5/00

(52) U.S. Cl. CPC G16H 30/20; G16H 10/40; G16H 50/20; G16H 50/30; G06T 7/11; G06T 2207/20008; G06T 3/40; G06T 5/009; G06T 2207/20081; G06T 2207/10056; G06T 7/0012

(57) ABSTRACT

A method and system for personalization of digital pathology analysis, may include: an image-analysis based diagnostics module, configured to extract at least one feature of a digital scan of a pathology slide of a patient, a human-machine interface module, configured to present the digital scan to a user for examination and at least one machine learning module, configured to produce at least one personalized suggestion according to the at least one extracted slide feature.

### FIELD OF THE INVENTION

The present invention relates generally to digital image analysis. More specifically, the present invention relates to personalization and optimization of digital pathology analysis.

### BACKGROUND OF THE INVENTION

Recent years have seen the rise of digital pathology, also referred to as virtual microscopy—the practice of pathology using scanned or imaged slides on a computer screen, rather than analyzing glass slides through a conventional microscope. This new modality has been driven by technological advancements in areas such as digital storage capacity, network speed and computer processing power. Whole slide image (WSI) scanners are used in many institutes around the world for training pathologists, research, tele-pathology, consultation, archiving and, most recently, for routine clinical diagnosis.

Digitization of pathology slides has also allowed to introduce new technologies that facilitate the pathologist's work, e.g., workflow software, measurement and annotation utilities and image sharing.

Computational Pathology, i.e., the automatic analysis of pathology images using advanced algorithms, has the potential to completely revolutionize the practice of pathology. Existent applications include automatic, and semi-automatic quantification of immunohistochemistry (IHC) slides, mitosis counting, slide registration (e.g., aligning Hematoxylin and Eosin (H&E) slides with corresponding IHC slides), and the like.

Various problems in computational pathology have been studied in the academic literature, including for example: image registration, 3D reconstruction, stain separation and standardization, feature extraction (e.g., glands, mitoses, and the like), cancer detection and cancer grading.

Commercially available software tools provide analysis of WSIs for both clinical diagnosis and research applications, although most existent clinical applications exhibit a limited scope, in the sense that they do not attempt to perform a full diagnosis. Instead, they produce a localized analysis, in tasks such as HC quantification, tissue segmentation and image registration.

Together with the growing adoption of digital pathology and automated diagnostic tools, pathology institutes face new challenges, namely, how to optimize the benefits of novel machine-learning based tools and customize them to the specific characteristics, requirements and preferences of each pathological institute and to each individual pathologist.

### SUMMARY OF THE INVENTION

Embodiments of the invention may include a system and a method for personalization and optimization of digital pathology analysis. Embodiments may collect data on the performance of digital pathology and automated diagnostic tools, and automatically adjust the behavior of these tools for optimal utilization and user experience in relation to individual pathologists and institutes.

Embodiments may include an image-analysis (IA) based computational diagnostics module that may be configured to receive at least one digital scan of at least one pathology slide associated with a pathological case and perform at least one algorithm of image analysis thereon to extract at least one feature of the at least one slide.

For example, the image analysis based module may be or may include any appropriate “Computer Aided Detection” (CADe), “Computer Aided Diagnosis” (CADx) “Computer Aided Diagnosis Quantification” (CADq) “Computer Aided Simple Triage” (CAST) or “CAD” software module as commonly referred to in the art. The CAD software may, for example be adapted to highlight conspicuous sections (e.g., pertaining to an anomaly or a disease) in an image (e.g., an image file) that may originate from a scanned pathology slide. The CAD software may provide one or more features pertaining to the slide and may thus offer input to support a decision taken by a human professional (e.g., a pathology expert).

Embodiments may further include a human-machine interface (HMI) module, configured to: present the at least one digital scan to a user for examination; receive from the user a selection of at least one viewing property; adjust the presentation of the at least one slide according to the selected viewing property; and produce a pathological report according to the examination.

Embodiments may further include at least one machine-learning (ML) module, configured to: produce at least one personalized suggestion according to the identity of the user and according to at least one of: an extracted slide feature, the selection of viewing property and the pathological report.

According to some embodiments, the produced suggestion may be selected from a list including at least one of: a first suggestion, relating to an assignment of at least one pathological case to a user, a second suggestion, relating to a preparation of at least one slide; a third suggestion, relating to an examination priority (e.g., of a slide, of a pathological case, etc.); a fourth suggestion, relating to a selection of at least one viewing property; a fifth suggestion, relating to a content of the pathological report; and a sixth suggestion, relating to a required second examination of the pathological case.

According to some embodiments, at least one extracted feature of a first slide associated with a first pathological case may be a first clinical feature, indicative of a first medical condition and wherein at least one extracted feature of a second slide may be a second clinical feature, associated with a second pathological case, indicative of a second medical condition. The third suggestion, relating to an examination priority (e.g., of a slide, of a pathological case, etc.) may include an assignment of a priority to the examination of the pathological cases, slides and/or locations in one or more slides, according to their respective indications of medical conditions.

Embodiments may further include a lab information sub-system (LIS), configured to maintain clinical data associated with at least one pathological case. The clinical data may include at least one of: clinical parameters associated with a patient; notes of a treating physician; a report of a radiologist; and a historical biopsy report.

The at least one ML module may be configured to produce the third suggestion, relating to an examination priority (e.g., of the pathological case), according to the clinical data of the LIS.

The LIS module may be configured to maintain workflow information associated with a plurality of users, comprising at least one of: a tissue type associated with the slide; a field of specialization of specific users of the plurality of users; a number of pathological cases assigned to each user of the plurality of users; and an availability of each user of the plurality of users. The term ‘user’ herein refers to a user of an embodiment of the present invention, and may typically refer to a pathological expert, examining a pathological case.

The at least one ML module may be configured to produce the first suggestion, relating to assignment of a pathological case to at least one user according to at least one of: the examination priority (e.g., of the pathological cases) and the workflow information of the LIS.

According to some embodiments, at least one pathological report may include at least one personal appearance preference of a user, regarding a parameter of appearance that may be associated with a pathological slide, and at least one extracted feature of a slide may be an appearance feature, associated with at least one element of the slide's appearance. The at least one ML module may be configured to produce the second suggestion, relating to a preparation of at least one slide, according to the user's identification and according to at least one of the personal appearance preference and appearance feature.

According to some embodiments, at least one pathological report may include at least one pre-ordering preference of a user, and at least one extracted feature of a slide may be a clinical feature, indicative of a medical condition. The at least one ML module may be configured to produce the second suggestion, relating to a preparation of at least one slide, according to the user's identification and according to at least one of the pre-ordering preference and clinical feature.

The computational diagnostics module may be configured to receive a plurality of digital scans associated with a pathological case, and at least one feature of at least one digital scan may include a region of interest (ROI) and at least one respective clinical feature. The at least one ML module may be configured to produce the fourth suggestion, relating to at least one viewing property, according to the user's identity and according to at least one of: the ROI, the at least one clinical feature and at least one selected viewing property.

According to some embodiments, at least one selected viewing property may include at least one of: an order of viewing of the plurality of digital scans by the user; a panning pattern of the presented digital scan by the user; a pattern of magnification of the presented digital scan by the user; and a selection of at least one of: brightness, contrast, color and gamma correction by the user.

According to some embodiments, at least one first pathological report of a user, associated with a first digital scan, may include a first ROI associated with a clinical feature, and a second digital scan of the same user may include a second ROI associated with the same clinical feature. The at least one ML module may be configured to produce the fifth suggestion, relating to a content of a second pathological report according to the user's identity and according to at least one of: the second ROI, the clinical feature, and the first pathological report.

The computational diagnostics module may be configured to: produce at least one clinical feature associated with at least one of: an ROI of at least one slide and an entire slide. The at least one ML module may be configured to: analyze the pathological report produced by the user, to locate one or more discrepancies between the pathological report and the clinical feature associated with the at least one ROI and produce the sixth suggestion, relating to a required additional examination of the at least one slide.

The at least one ML module may be configured to: obtain historic data associated with a plurality of historic diagnoses of pathological cases from the LIS, produce at least one classification model of the historical data, associating each class with a probability of error; predict a probability of error for a new pathological case, beyond the plurality of historic cases; and produce the sixth suggestion, relating to a required additional examination of the pathological case according to the predicted probability of error.

The historical data may include at least one of: parameters of the diagnosis process; a clinical feature of at least one slide associated with the pathological case; clinical parameters associated with the respective patient; and parameters of an error in the diagnosis process.

Embodiments of the present invention may include a system for personalization of digital pathology analysis. Embodiments of the system may include: an image analysis based computational diagnostics module, configured to receive at least one digital scan of at least one pathology slide associated with a pathological case and perform at least one algorithm of image analysis thereon to extract at least one feature of the at least one slide; a human-machine interface module, configured to present the at least one digital scan to a user for examination; and at least one machine-learning module, configured to produce at least one personalized suggestion according to the at least one extracted slide feature.

According to some embodiments, the personalized suggestion may include, for example: an assignment suggestion, a preparation suggestion, a prioritization suggestion, a viewing suggestion, a pathological report suggestion and a second examination suggestion.

According to some embodiments, the HMI may be configured to receive from the user a selection of at least one viewing property and the at least one ML module may be configured to produce the at least one personalized suggestion according to an identity of the user and according to at least one of: the extracted slide feature and the selected viewing property.

According to some embodiments, the HMI may be configured to produce a pathological report according to the examination. The at least one ML module may be configured to produce the at least one personalized suggestion according to the identity of the user and according to at least one of: the extracted slide feature, the selected viewing property and the pathological report.

According to some embodiments, a first extracted feature of a first slide may be a first clinical feature, indicative of a first medical condition and a second extracted feature of a second slide may be a second clinical feature, indicative of a second medical condition. The personalized prioritization suggestion may include assignment of an examination priority to the slides according to their respective indications of medical conditions.

Embodiments of the system may include a lab information sub-system, configured to maintain clinical data. The maintained clinical data may include, for example: clinical parameters associated with a patient; notes of a treating physician; a report of a radiologist; and a historical biopsy report. The at least one ML module may be configured to produce the personalized prioritization suggestion according to the clinical data of the LIS.

According to some embodiments, the LIS may be configured to maintain workflow information. The maintained workflow information may include, for example: a tissue type associated with the slide, a field of specialization of specific users, a number of pathological cases assigned to each user and an availability of each user. The at least one ML module may be configured to produce the personalized assignment suggestion according to at least one of: the examination priority and the workflow information.

According to some embodiments, at least one pathological report may include at least one personal appearance preference of a user and at least one extracted feature may be an appearance feature associated with a pathology slide. The at least one ML module may be configured to produce the personalized preparation suggestion according to the user's identification and according to at least one of the personal appearance preference and the appearance feature.

According to some embodiments, at least one pathological report may include at least one pre-ordering preference of a user and at least one extracted feature of a slide may be a clinical feature, indicative of a medical condition. The at least one ML module may be configured to produce a personalized preparation suggestion according to the user's identification and according to at least one of the pre-ordering preference and clinical feature.

According to some embodiments, at least one extracted feature may include an ROI and a respective clinical feature. The at least one ML module may be configured to produce a personalized viewing suggestion according to the user's identity and according to at least one of: the ROI, the at least one clinical feature and at least one viewing property.

According to some embodiments, the at least one ML module may be configured to: produce an examination story of a pathologist's examination of a specific pathology slide; extract one or more personal viewing property preferences, pertaining to the specific pathologist; and produce a personalized viewing suggestion according to the user's identity and according to the examination story.

According to some embodiments, the at least one viewing property may include, for example: an order of viewing of the plurality of digital scans by the user, a panning pattern of the presented digital scan by the user; a pattern of magnification of the presented digital scan by the user; and a selection of at least one of: brightness, contrast, color and gamma correction by the user.

According to some embodiments, at least one first pathological report of a user, associated with a first digital scan, may include a first ROI associated with a clinical feature, and a second digital scan may include a second ROI associated with the same clinical feature. The at least one ML module may be configured to produce the personalized report suggestion according to the user's identity and according to at least one of the second ROI, the clinical feature, and the first pathological report.

According to some embodiments, the extracted feature may be a clinical feature associated with at least one of an ROI of at least one slide and an entire slide. The at least one ML module may be configured to: analyze the pathological report to determine one or more discrepancies between the pathological report and the extracted clinical feature; and produce a personalized second examination suggestion based on the determined one or more discrepancies.

According to some embodiments, the at least one ML module may be configured to: obtain historical data associated with a plurality of historic diagnoses of pathological cases from an LIS; produce at least one classification model of the historical data, associating each class with a probability of error; predict a probability of error for a new pathological case, beyond the plurality of historic cases; and produce a second examination suggestion according to the predicted probability of error.

According to some embodiments, the historical data may include at least one of: parameters of the diagnosis process; a clinical feature of at least one slide associated with the pathological case; clinical parameters associated with the respective patient; and parameters of an error in the diagnosis process.

Embodiments of the present invention may include a method for personalization of digital pathology analysis by at least one processor. Embodiments of the method may include: receiving at least one digital scan of at least one pathology slide associated with a pathological case; performing at least one algorithm of image analysis on the received digital scan to extract at least one slide feature; presenting, on an HMI the at least one digital scan to a user for examination; and producing, by at least one ML module, at least one personalized suggestion according to the extracted slide feature.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a flow diagram, depicting an example for a workflow at a diagnostic pathology laboratory or institute;

FIG. 2 is a block diagram, depicting a computing device, which may be included within an embodiment of a system for personalization and optimization of digital pathology analysis, according to some embodiments;

FIG. 3 is a schematic block diagram, depicting a process of IA-based computational diagnostics, which may be implemented by a system for personalization and optimization of digital pathology analysis, according to some embodiments;

FIG. 4 is a schematic block diagram, depicting a system for personalization and optimization of digital pathology analysis, according to some embodiments;

FIG. 5 is a block diagram, depicting a pre-analytics module, which may be included within an embodiment of a system for personalization and optimization of digital pathology analysis, according to some embodiments;

FIG. 6 is a block diagram, depicting a triage module, which may be included within an embodiment of a system for personalization and optimization of digital pathology analysis, according to some embodiments;

FIG. 7 is a block diagram, depicting a decision support module, which may be included within an embodiment of a system for personalization and optimization of digital pathology analysis, according to some embodiments;

FIG. 8 is a block diagram, depicting a quality control (QC) module, which may be included within an embodiment of a system for personalization and optimization of digital pathology analysis, according to some embodiments; and

FIG. 9 is a flow diagram, depicting a method for personalization and/or optimization of digital pathology analysis, according to some embodiments.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining.” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

State of the art systems of digital pathology analysis include implementation of advanced computational algorithms, such as Deep Learning and Computer Vision, to detect malignant and other clinical features in examined specimens, including for example prostate core needle biopsies. Such systems may, for example, implement an automated Quality Control (QC) mechanism for all samples that may have been diagnosed as benign, and may produce an alert whenever a discrepancy between an automated analysis and an experts diagnosis is identified, to prompt a second human review.

Embodiments of the present invention may include a method and a system for personalization and optimization of digital pathology analysis, according to characteristics and policies associated with specific pathologists and/or pathology institutes. For example, embodiments of the present invention may include optimization and/or personalization of: computer aided diagnostic processes and/or tools; triage and/or clinical prioritization processes and/or tools; pre-analysis and/or preordering processes and/or tools, and the like, as elaborated herein.

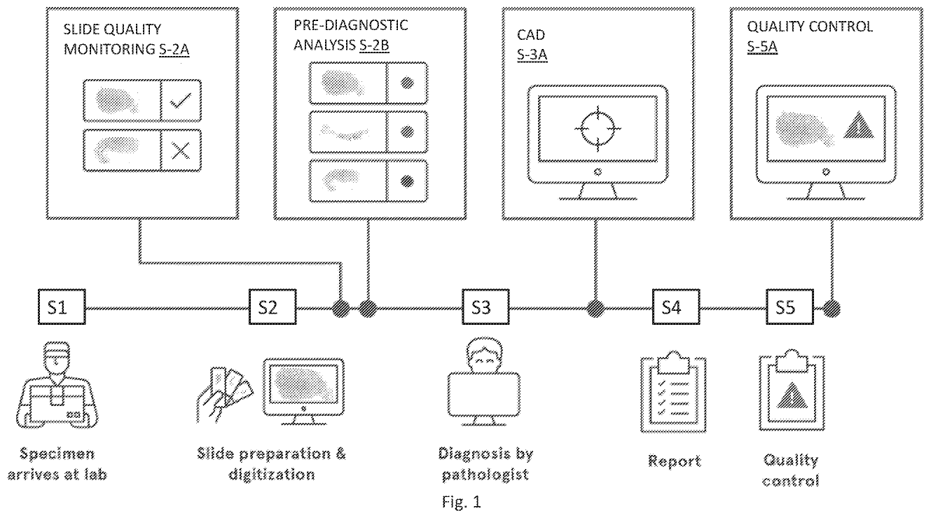

Reference is now made to FIG. 1, which is a flow diagram, depicting an example for a workflow at a diagnostic pathology institute. The workflow of FIG. 1 depicts processes that may normally be performed in pathology institutes as known in the art. Embodiments of the present invention may be adapted to improve (e.g., by personalizing and/or optimizing) processes depicted in FIG. 1, as elaborated herein.

As shown in step S1, a specimen (e.g., a sample such as a tissue biopsy that may have been ordered by a physician) may arrive at the pathological institute and may be subject to pathological analysis, as described herein.

In step S2, one or more samples may be processed, to prepare and digitally scan or image (e.g. produce an image of) one or more slides, as normally performed in pathological institutes and as known in the art. The processing of samples may include a plurality of steps, such as: sampling, fixation and cutting of the specimen, to obtain one or more slices that are surmountable on one or more glass slides; staining the slides according to requirements of the present clinical case; and performing high-resolution scanning of the slides, e.g. by a WSI scanner, to obtain high resolution images.

Embodiments of the present invention may optimize and/or personalize slide preparation steps elaborated above, as explained herein. Moreover, some embodiments of the present invention may produce a notification or a feedback to a sample processing system (e.g., to the WSI scanner, to the slicer, etc.) regarding a desired parameter (e.g., resolution of a WSI scan) or a change thereof.

For example, as shown in step S-2A, embodiments of the invention may employ digital image processing algorithms to monitor the quality of images, e.g. scanned slides, and mark scanned slides that do not comply predefined quality criteria. Such global criteria may include, for example: compliance of the processed sample to requirements of the present clinical case (e.g., performance of specific staining), quality of the glass slide (e.g., amount of dirt, dust, cracks or air bubbles), thickness of the tissue slice, amount of tissue folds and holes in the slice, and features of appearance of the slide (e.g., uniformity of the staining, focus of the scanned image, lighting and/or brightness of the scanned image, contrast of the scanned image, and the like, hereinafter appearance features).

Embodiments of the invention may use ML algorithms to learn preferences or requirements of one or more specific pathologists and/or policies of specific pathology institutes and examine or monitor the quality of scanned slices to accommodate such personal criteria of requirements and preferences.

For example, embodiments of the invention may receive at least one scanned slide and at least one respective requirement made by a specific pathologist in relation to the scanned slide, and may be trained to learn that the specific pathologist and/or a policy of a pathology institute may require specific features of slides (e.g., types of slides, qualities of staining, etc.) for specific clinical cases. Embodiments of the invention may consequently produce a suggestion (e.g., as an electronic message) to a physical entity in the pathology institute, such as a sample processing system (e.g., a WSI scanner, a slicer, etc.), to provide the required slide at the required quality.

In yet another example, as shown in step S-2B, embodiments of the present invention may employ IA-based computational diagnostics algorithms (e.g., a CAD software module) to perform a first reading of the at least one scanned slide and detect one or more slides that may include visual or morphological features of clinical significance (hereinafter clinical features). Embodiments of the invention may consequently optimize and/or personalize the process of pre-diagnosis.

For example, embodiments may be trained to perform an optimized triage of pathological cases. Embodiments may receive at least one slide and may detect one or more ROIs that may include clinical features. Embodiments may include an ML module, adapted to be trained (e.g., by receiving supervisory information from a human pathologist) to sort the scanned slides according to the detected ROIs and clinical features. In such embodiments, slides and ROIs of high clinical significance (e.g., slides including malignant cells) are presented to the pathologist before slides and ROIs of low clinical significance (e.g., slides including inflammation), enabling a human pathologist to focus on slides and ROIs of high significance.

In another example, embodiments may be trained to perform personalized routing of specific cases to specific examining pathologists. Embodiments may detect one or more ROIs that may include clinical features and may receive personal data relating to human pathologists in the pathology institute. Embodiments may include an ML module, adapted to receive personal information (e.g., from a database related to the pathology institute) that may include, for example: the identity of available experts in the pathology lab or institute, their workload, their expertise, their rate of success, their probability of error and the like. Embodiments may train the ML module to route or assign specific cases to specifically selected experts according to at least one of the personal information, the pathological institute's policy and the detected clinical features.

Such embodiments may, for example be configured to maintain a task list (e.g., a table in a database associated with the institute), that may associate pathological analysis tasks with pathological experts of the pathological institute or laboratory. Embodiments may assign or route cases to pathological experts by updating entries in the task list (e.g., assign a specific case number to a specific expert). For example, embodiments may assign, or route ‘simple’ cases to junior pathologists, and more subtle cases to experienced personnel, to optimize the yield and success rate of the pathology institute and present the assigned cases of each pathologist on a user interface.

Specific pathologists may order specific additional slides (e.g., IHC slides) in view of specific clinical findings. In yet another example, embodiments may be trained to perform personalization of pre-ordering of additional tests. Embodiments may receive at least one slide and may detect one or more clinical features on the slide. Embodiments may include an ML module, adapted to be trained (e.g., by receiving supervisory information from a human pathologist) to learn which additional tests should be ordered by each pathologist given specific slide appearance and/or clinical features. Such embodiments may thus personalize the process of pre-ordering and may improve the efficiency and yield of the pathology lab.

As shown in step S3, a pathologist may examine the one or more scanned slides to detect clinical features therein. Embodiments of the invention may present the slides on any appropriate HMI module to a selected pathologist.

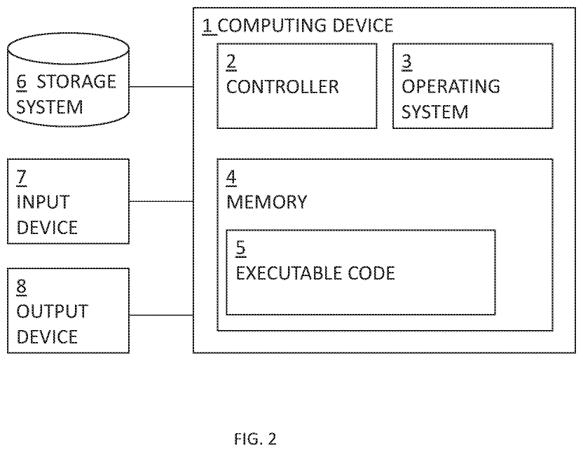

For example, the HMI may be or may include a Digital Pathology Workflow (DPW) module as known in the art. Additionally, or alternatively, the HMI may be implemented on any appropriate computing device (e.g., element 1 of FIG. 2) that may include an output device (e.g., element 8 of FIG. 2, such as a screen or monitor) and/or an input device (e.g., element 7 of FIG. 2 such as a keyboard, a mouse and the like). Embodiments of the invention may present the slides on any appropriate HMI module to the selected pathologist (e.g., on a computing device associated with the selected pathologist) as elaborated above, according to the personalized triage, and according to the optimized order of significance.

As explained herein, embodiments of the invention may employ CAD software algorithms to extract and/or produce one or more features pertaining to one or more examined slides. Embodiments of the invention may improve currently available IA-based algorithms (e.g., based on proprietary and/or commercially available CAD software modules) by providing personalized computer aided diagnosis. For example, embodiments of the invention may highlight specific ROIs that may include clinical features to the pathologist during the process of examination, according to the pathologist's preferences.

For example, an embodiment may receive at least one slide and may detect one or more clinical features on the slide. The embodiment may include an ML module that may receive data relating to a specific pathologist's work flow and may be adapted to learn an order of examination that may be preferred by the specific pathologist (e.g., start with an overall view or start by zooming-in on specific ROIs). The embodiment may thus present the scanned slide according to the personalized order of examination, to save time by guiding the human pathologist through the examined specimen according to the pathologist's preferred work flow.

As shown in step S4, the pathologist may produce a report, including his or her examination of the at least one scanned slide. Embodiments may include an ML module, configured to receive at least one slide and a respective at least one report produced by a specific human pathologist, and learn a format of a pathological report, that may be preferred by the specific pathologist in view of the respective slide. Embodiments may produce a report having a similar format, according to the concurrent examination of step S-3A, and thus save time and improve the yield of the pathological institute.

As shown in step S-3A, embodiments of the invention may be configured to perform a concurrent examination of the scans of the present pathological case, parallel to the examination performed by the pathological expert.

Embodiments may include an ML module, configured to receive, as input: at least one slide and supervisory information from an expert pathologist.

The ML module may include a medical image analysis module, that may be trained to identify one or more pathological conditions according to the received input, as known in the art.

In some embodiments, the ML module may further receive data corresponding with the medical background or history of the patient (e.g., age, gender, results of laboratory tests, previous identified pathologies, etc.), and may be trained to predict a probability of a specific malignancy according to the medical background data. For example, the ML module may include a clustering model, adapted to cluster a plurality of examined pathological cases according to a plurality of medical background features (e.g., age, previous test results, etc.) and predict a probability a specific medical finding (e.g., a malignant cell in a sampled biopsy) according to the medical background features and supervisory data (e.g., labeling of slides as OK or not) received from a pathological expert.

According to some embodiments, the ML module may further be configured to receive data corresponding with at least one condition of the pathologist's examination (e.g., type of clinical case, number of scanned slides, clinical features detected in one or more scanned slides, appearance features of one or more scanned slides, time of day, number of examinations performed that day, etc.).

As shown in step S5, embodiments of the present invention may detect discrepancies between a first report produced by a first pathologist and a second report, produced by the at least one ML module during the concurrent examination of step S-3A, as elaborated herein further below. Upon detection of such a discrepancy, embodiments may be configured to prompt a requirement for a second examination by a second human pathologist.

For example, embodiments may provide to the second pathologist (e.g., via an email, via a digital pathology workflow module as known in the art, and the like) the first report (of the first pathologist) and the second report (of the ML module), highlight the discrepancies between them, and assign (e.g., by updating a task list on a database of the pathological institute) a second examination of the pathological case to the second pathologist.

If the second reading or examination verifies that an error has been made during the first pathologist's examination, embodiments of the invention may store information relating to circumstances or conditions of the erroneous examination.

Embodiments of the invention may produce an ML model that may include clusters or classes of instances of erroneous results, and may associate additional, new examination cases with a probability of error, according to the ML model. Embodiments of the invention may then produce a suggestion (e.g., as an electronic message) to perform a second reading of at least one slide related to a pathological case, based on a high probability of error, as elaborated herein.

Reference is now made to FIG. 2, which is a block diagram depicting a computing device, which may be included within an embodiment of a system for personalization and optimization of digital pathology analysis, according to some embodiments.

Computing device 1 may include a controller 2 that may be, for example, a central processing unit (CPU) processor, a chip or any suitable computing or computational device, an operating system 3, a memory 4, executable code 5, a storage system 6, input devices 7 and output devices 8. Controller 2 (or one or more controllers or processors, possibly across multiple units or devices) may be configured to carry out methods described herein, and/or to execute or act as the various modules, units, etc. More than one computing device 1 may be included in, and one or more computing devices 100 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of Computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short-term memory unit, a long-term memory unit, or other suitable memory units or storage units. Memory 4 may be or may include a plurality of, possibly different memory units. Memory 4 may be a computer or processor non-transitory readable medium, or a computer non-transitory storage medium, e.g., a RAM.

Executable code 5 may be any executable code, e.g., an application, a program, a process, task or script. Executable code 5 may be executed by controller 2 possibly under control of operating system 3. For example, executable code 5 may be an application that enforces security in a vehicle as further described herein. Although, for the sake of clarity, a single item of executable code 5 is shown in FIG. 2, a system according to some embodiments of the invention may include a plurality of executable code segments similar to executable code 5 that may be loaded into memory 4 and cause controller 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Content may be stored in storage system 6 and may be loaded from storage system 6 into memory 4 where it may be processed by controller 2. In some embodiments, some of the components shown in FIG. 2 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a USB device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of CPUs or any other suitable multi-purpose or specific processors or controllers (e.g., controllers similar to controller 2), a plurality of input units, a plurality of output units, a plurality of memory units, and a plurality of storage units.

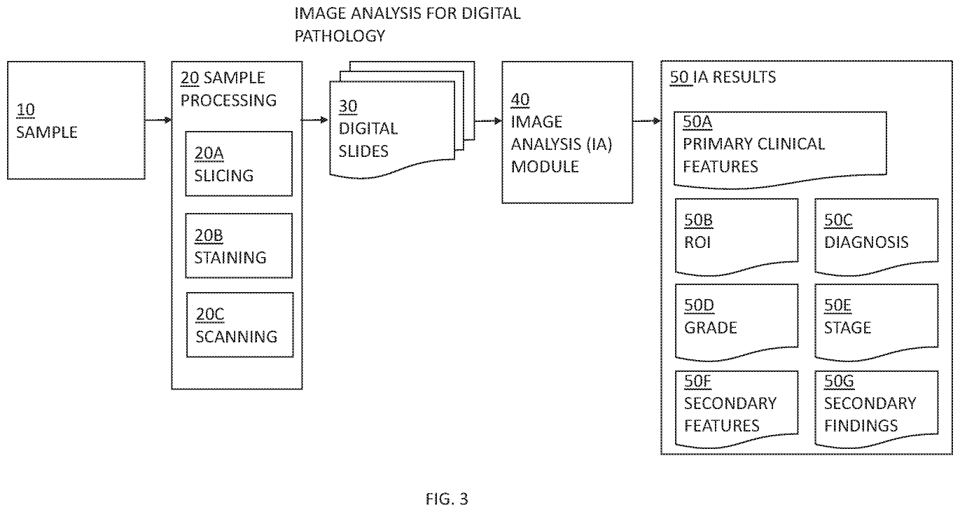

Reference is now made to FIG. 3, which is a schematic block diagram, depicting a process of IA-based computational diagnostics or CAD, which may be implemented by a system for personalization and optimization of digital pathology analysis, according to some embodiments.

As elaborated herein, in relation to FIG. 1, a workflow of a pathological institute may include receiving a specimen or a sample 10 of a tissue, associated with a specific pathological case. The tissue may be processed by one or more processing modules, according to predefined requirements, associated with the type of the tissue and a suspected clinical condition, to obtain one or more digitally scanned slide images 30. For example, sample 10 may be: sliced to a predefined thickness by a slicing module 20A; stained according to predefined clinical requirements or protocols by a staining module 20B, where the predefined clinical requirements may be dictated according to specifications of the clinical case (e.g., a suspected type of cancer); and digitally scanned according to predefined configurations (e.g., resolution, brightness, contrast, etc.) by a high-resolution scanning module (e.g., a WSI scanner) 20C.

IA computational diagnostics module (hereinafter IA module) 40 may receive one or more digitally scanned slides 30 from the one or more processing modules 20 (e.g., 20A, 20B and 20C) and may employ one or more algorithms to extract one or more clinical features, and produce one or more diagnostic result 50 (e.g., 50A, 50B, 50C, 50D, 50E, 50F and 50G), as known in the art. The one or more diagnostic result 50 of IA module 40 may consequently be utilized by an embodiment of a system for personalization and optimization of digital pathology analysis.

According to some embodiments, IA module 40 may be implemented as a computational module, that may be executed by a processor (e.g., element 2 of FIG. 2), to produce the one or more diagnostic results 50. For example, IA module 40 may be or may include an artificial-intelligence based application or software module, adapted to train a neural network (NN), such as a deep NN, a convolutional NN and/or any other computational module known in the art, to extract at least one clinical feature of the scanned slide.

For example, IA module 40 may be configured to extract or detect at least one of: a primary clinical feature 50A, that may indicate a clinical condition of primary importance or priority (e.g., existence of a malignant tumor, indication of a perineural invasion, etc.), including for example: a morphological or cellular feature (e.g., a shape of one or more cells, a size of one or more cells, and the like), a pattern of a stain or dye absorbed by the examined tissue, etc.; an ROI 50B, that may be suspected to include an extracted clinical feature 50A, and should therefore be studied carefully by an expert pathologist; a suggested diagnosis 50C (e.g., a malignant tumor of a particular type), corresponding to the extracted primary clinical feature 50A and detected ROI 50B; a grade 50D and/or a stage 50E of a cancerous tumor, as per the suggested diagnosis 50C, as known in the art of oncological medicine; a secondary clinical feature 50F that may indicate a clinical condition of secondary importance or priority (e.g., a pre-cancerous condition); and a suggested secondary finding 50, corresponding to the extracted secondary clinical feature 50F (e.g., an inflammation, a pre-cancerous condition, and the like).

An embodiment of the present invention may utilize one or more results 50 (e.g., 50A through 50G) to optimize the process of diagnosis by a pathology expert (e.g. by controlling and modifying sample pre-processing 20, prioritizing scanned slides according to primary and secondary findings and performing triage of pathological cases) according to each pathologist's work method and preference, and according to policies of the pathological institute, as explained herein.

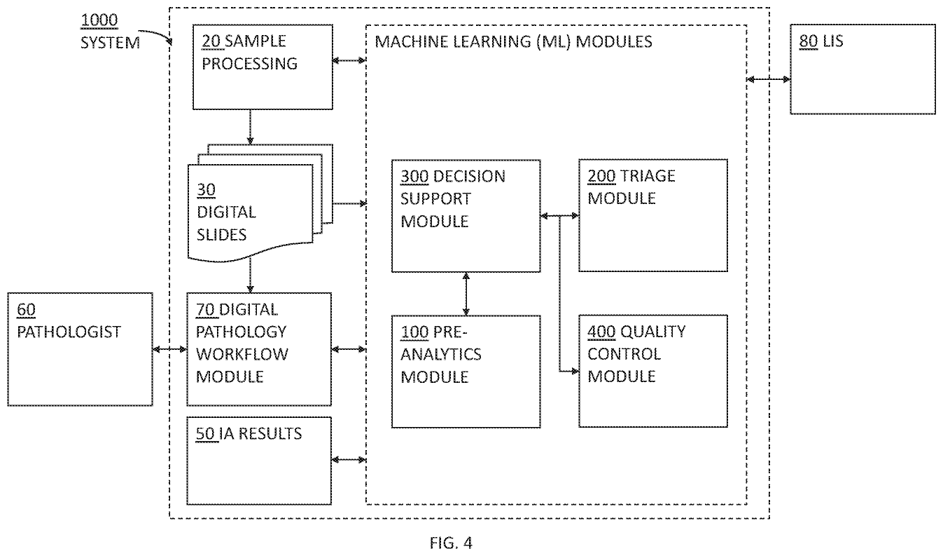

Reference is now made to FIG. 4, which is a schematic block diagram, depicting a system 1000 for personalization and optimization of digital pathology analysis, according to some embodiments.

System 1000 may include an image-analysis based computational diagnostics module (e.g. element 40 of FIG. 3), configured to receive at least one digital scan (e.g., a high-resolution image file, as known in the art) of at least one pathology slide associated with a pathological case and perform at least one algorithm of image analysis thereon to extract at least one feature or result 50 of the at least one slide, as elaborated above, in relation to FIG. 3.

System 1000 may include an HMI module, such as a DPW module 70, as known in the art. DPW 70 may be implemented as a software module (e.g. a software application or process) and may be executed by a computing device (e.g. element 1 of FIG. 2).

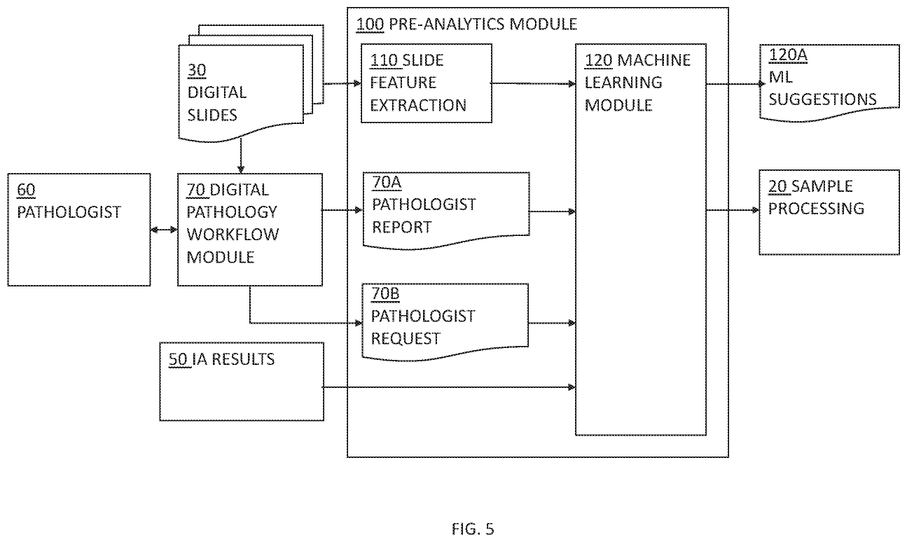

DPW 70 may be configured to facilitate a user's (e.g., an expert pathologist's) inspection or examination of a suspected specimen. For example, DPW 70 may; present or display (e.g., on a screen) the at least one digital scan to a user for examination; enable the user to select, or receive from the use a selection of, at least one viewing property (e.g., zoom (magnification level) in/out of the presented image, panning of the presented image, brightness, gamma correction, contrast, and the like); store (e.g., on element 6 of FIG. 2) at least one action or selection performed by the user; adjust the presentation of the at least one slide (e.g., apply a high-pass filter, change the contrast, etc.) according to the at least one selected viewing property, and produce a pathological report (e.g., element 70A of FIG. 5) according to the examination, as known in the art of digital pathology workflow.

In some embodiments, system 1000 may interface with or include an LIS module 80. As known in the art, LIS module 80 may include a database, and may maintain or store clinical data or workflow information associated with one or more pathological cases, including for example: clinical parameters associated with a patient (e.g., lab test results, gender, age, weight, prescriptions, chronic diseases, family history, etc.); notes of a treating physician (e.g., notes that pertain to a suspected diagnosis); a report of a radiologist; a historical biopsy report associated with the patient; a type of tissue associated with the slide; a field of specialization of a pathologist (e.g. a specific pathologist of a plurality of pathologists); a number of pathological cases assigned to each pathologist; history of circumstances of pathological examinations (e.g. time of day, general workload, time spent on each examination, and the like) per each pathologist; and an availability of each pathologist of the plurality of pathologists.

System 1000 may include one or more ML modules (e.g. included within elements 100, 200, 300 and 400, as explained herein), configured to produce at least one personalized suggestion according to the identity of the user (e.g. a specific pathologist) and according to at least one of: an extracted slide feature or result 50; a selection of a viewing property; a policy of the pathology laboratory or institute; clinical data stored or maintained within the LIS; and a pathological report, as explained herein.

For example: Triage module 200 may produce a first personalized suggestion, such as an assignment suggestion, relating to an assignment of at least one pathological case to a specific user (e.g. a specific expert pathologist). In another example, pre-analytics module 100 may produce a second personalized suggestion, such as a preparation suggestion, relating to a process of preparation of at least one slide. In another example, triage module 200 may produce a third personalized suggestion, such as a prioritization suggestion, relating to an optimized prioritization of the pathological case. In another example, decision support module 300 may produce a fourth personalized suggestion, such as a viewing suggestion, relating to a selection of at least one viewing property. In another example, decision support module 300 may produce a fifth personalized suggestion, such as a pathological report suggestion, relating to a personalized content of the pathological report. In yet another example, QC module 400 may produce a sixth personalized suggestion, such as a second examination suggestion, relating to a required second examination of the pathological case.

The content and nature of each suggestion is elaborated herein, in relation to each of modules 100, 200, 300 and 400.

Each of modules 100, 200, 300 and 400 may include an ML model that may be configured or trained to produce a respective suggestion, as explained herein. Each of the ML models may be: a supervised model (such as Linear or Logistic Regression, Random Forest. Gradient Boosting, Deep Learning, and the like), in which the data collected to train the model may be labelled, or an unsupervised model (such as a Nearest Neighbor model), in which the data may not be labelled, according to the relevant context. For example, one or more ML models of modules 100, 200, 300 and 400 may be or may include an artificial-intelligence based application or module, adapted to train a neural network such as a deep NN, a convolutional NN and/or any other computational module known in the art.

A neural network, e.g. a neural network implementing machine learning, may refer to an information processing paradigm that may include nodes, referred to as neurons, organized into layers, with links between the neurons. The links may transfer signals between neurons and may be associated with weights. A NN may be configured or trained for a specific task, e.g., pattern recognition or classification. Training a NN for the specific task may involve adjusting these weights based on examples. Each neuron of an intermediate or last layer may receive an input signal, e.g., a weighted sum of output signals from other neurons, and may process the input signal using a linear or nonlinear function (e.g., an activation function). The results of the input and intermediate layers may be transferred to other neurons and the results of the output layer may be provided as the output of the NN. Typically, the neurons and links within a NN are represented by mathematical constructs, such as activation functions and matrices of data elements and weights. A processor, e.g. CPUs or graphics processing units (GPUs), or a dedicated hardware device may perform the relevant calculations.

According to some embodiments, system 1000 may enable a user to provide feedback (e.g. via DPW 70) to optimize the training of any of the ML models, thus facilitating continuous improvement and fine-tuning of the production of any of the produced suggestions.

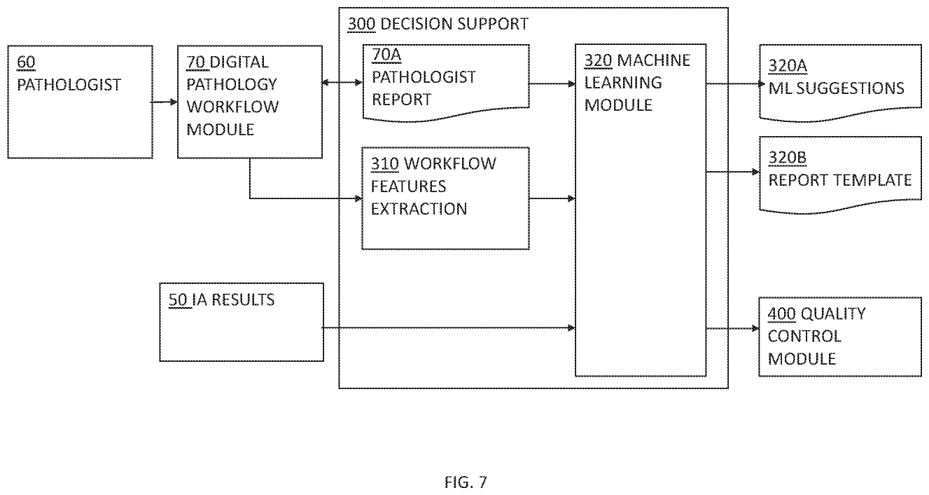

For example, decision support module 300 may produce a personalized pathological report suggestion (e.g., ML suggestion element 320A of FIG. 7), relating to a personalized content of the pathological report, and may also produce a report template, as discussed below in relation to FIG. 7. A pathologist may apply at least one amendment to the report template, and decision support module 300 may use this amendment as feedback (e.g., supervisory feedback) for training its respective ML model, and fine-tune the template for future, similar medical cases.

Reference is now made to FIG. 5, which is a block diagram, depicting a pre-analytics module 100, which may be included within an embodiment of a system for personalization and optimization of digital pathology analysis, according to some embodiments.

The term pre-analytics is used herein to refer to any actions that may be applied to a scanned slide prior to handling of the slide by a designated human pathologist. For example, a pre-analytics process may include: extraction of appearance features; analysis of the appearance features according to global requirements (e.g., detect whether a quality of a specific slide does not match a global requirement); analysis of the appearance features according to a pathology institute's policy or a specific pathologist's preferences (e.g., detect whether a quality of a specific slide does not match the pathologist's preferences); reordering of slides according to a specific pathologist's preferences and/or according to an institute's policy (e.g., when a quality of a scanned slide is inadequate); and pre-ordering of additional slides according to a specific pathologist's preferences and/or according to an institute's policy and/or according to global requirements.

Computational analysis may offer an efficient validation that the slide has been prepared and scanned in a quality that allows pathological examination. According to some embodiments, pre-analytics module 100 may automatically learn at least one preference or requirement of slide quality per at least one specific pathologist and may adapt to changing conditions in the pathology institute.

For instance, a staining protocol used in one institute might result in slides that would be considered badly stained in another. In another example, staining may gradually deteriorate over time. Therefore, a monitoring tool that corresponds with predefined parameters (e.g., stain colors properties) would be impractical or difficult to use, whereas a monitoring system that adjusts itself to the preferences of each pathologist and to the slide quality standards in each institute may be significantly more practical and useful.

Pre-analytics module 100 may be configured to monitor appearance properties including, for example: scanning quality parameters, such as: focus, contrast, colors, tissue detection (scanners scan only the parts of the slide that contain tissue; tissue detection, whether performed manually or automatically, might miss some tissue areas); staining quality and consistency parameters, such as: one or more color of each stain type (e.g., Hematoxylin and Eosin in Hematoxylin and Eosin slides, or other stains in IHC slides), the amount of stain and the uniformity of the staining throughout the slide; and tissue cutting and mounting parameters, such as the width of the cut, consistency of the width, tissue folds, tears and holes, overlap of tissue slices, cracks or dirt in the glass slide.

Pre-analytics module 100 may include a slide feature extraction module 110, configured to receive one or more scanned slides, and extract at least one appearance feature associated with at least one element of the slide's appearance. For example, feature extraction module 110 may detect folds and holes in the scanned slide, determine a thickness of the sliced tissue, detect properties of staining (e.g., stain colors and intensity), detect properties of scanning (e.g., focus, colorization, contrast, etc.) of the slide, etc.

In some embodiments, pre-analytics module 100 may receive predefined parameters associated with one of: a policy of a pathology laboratory or institute and a global requirement of the slide's appearance and produce at least one personalized preparation suggestion (e.g., ML suggestions element 120A), based on the at least one extracted appearance feature relating to the preparation of at least one examined slide.

In some embodiments, a policy of a pathology institute may be implemented as a table of requirements stored at a database (e.g., element 6 of FIG. 2) of the pathology institute, an may associate specific malignancies with specific handling requirements. For example, the requirements table may dictate that specific types of suspected clinical cases should be treated with specific types of stains that should produce specific colorization characteristics.

A processor (e.g., element 2 of FIG. 2) associated with, or included within feature extraction module 110 may read the table of requirements and may identify one or mom cases that do not comply with the dictated policy. For example, feature extraction module 110 may identify a condition in which the colorization does not comply with a requirement as dictated by the policy (e.g., due to improper and/or insufficient staining).

Feature extraction module 110 may produce a respective personalized preparation suggestion 120A to at least one sample processing module 20, e.g., to amend the requirement of the slide's appearance (e.g. amend the staining of the slide) or to produce a new scanned slide according to the dictated policy, before propagating the sample to examination by a pathologist, to reduce turnaround time and pathologist workload.

In some embodiments, preparation suggestion 120A may be sent automatically, as an electronic message to an automated sample processing module 20, that may perform the required amendment automatically. Additionally, or alternatively, preparation suggestion 120A may be sent to a human practitioner (e.g., as an email containing the required amendment) that may perform the required actions or amendments accordingly.

In some embodiments, the pathological institute's policy may be dynamically influenced by parameters associated with a workflow of the institute. For example, pre-analytics module 100 may enable a user to enter (e.g. via DPW 70) at least one parameter (e.g. a price of a staining process). The institute's policy may dictate that the overall expenditure on a specific diagnosis should not exceed a predefined sum, and the decision of whether to perform an additional process (e.g. produce an additional stained slide) may be influenced by the entered parameter and the institute's policy.

In another example, a global requirement (e.g., not related to a specific pathological institute) may determine that a thickness of a sliced specimen may not surpass a predefined threshold or may not include folds or holes. Feature extraction module 110 may identify one or more cases that do not comply with these requirements and ML module 120 may consequently produce a respective preparation suggestion 120A to at least one sample processing module 20 (e.g., as an electronic message) or to a human practitioner (e.g., as an email, as elaborated above), to amend the sliced sample before propagating the sample to examination by a pathologist.

In some embodiments, ML module 120 may receive at least one feature, associated with a specific slide from feature extraction module 110, an IA result 50 and at least one request 70B from a specific pathologist, relating to the same slide. ML 120 may be configured to learn therefrom at least one personal appearance preference of a specific user (e.g. a pathologist), that may be related to one or more parameters of an appearance or a preparation of a slide. The appearance parameter may include, for example: a coherence or quality of a staining process, a thickness of a slice, a scanning resolution of a slice, a contrast of a scanned image of a slice, a sharpness of a scanned image of a slice, and the like.

Pre-analytics module 100 may consequently produce at least one preparation suggestion 120A to at least one sample processing module 20 according to the user's identification, and according to at least one of: the learnt personal appearance preference, an appearance feature extracted by module 110, and an IA result 50.

For example, a pathologist 60 may not be able to complete a diagnosis or may produce at least one pathological report 70A or request 70B via DPW 70 that may include at least one personal appearance preference relating to a pathological slide. For example, a pathologist may require a specific amendment (e.g., a higher resolution of scanning) to properties of a slide that may not have been sliced, scanned or stained properly.

ML module 120 may include a prediction model (e.g. a supervised model or an unsupervised model) that may receive at least one of: the specific expert pathologist's identity or an identity of a plurality of experts, at least one appearance feature extracted by module 110 (e.g. level of focus) and at least one clinical feature or result 50 (e.g. spiculation of a cell). ML module 120 may be trained according to historical requests made by the one or more expert pathologists to determine the personal appearance preferences of the one or more expert pathologists and predict whether an amendment of one or more scanned slides is required, and what the nature of the amendment should be (e.g., rescan with improved level of focus).

In some embodiments, ML module 120 may be configured to learn at least one pre-ordering preference of a specific user (e.g. a pathologist) and pre-analytics module 100 may consequently produce at least one personalized preparation suggestion 120A to at least one sample processing module 20 according to the user's identification, and according to at least one of: the learnt pre-ordering preference and an IA result 50.

For example, a pathologist 60 may produce at least one pathological report 70A or request 70B via DPW 70. The report 70A or request 70B may include at least one pre-ordering preference, relating to a pathological slide. For example, a specific pathologist may require ordering an additional slide, including a specific type of stain (e.g. an IHC stain) for a pathological case that may be characterized by a specific diagnosis 50C, grade 50D and stage 50E.

ML module 120 may include a prediction model (e.g. a supervised model or an unsupervised model) that may receive at least one of: the specific expert pathologist's identity or an identity of a plurality of experts and at least on clinical feature or result 50, and may be trained according to historical requests made by the one or more expert pathologists to determine the pre-ordering preferences of the one or more expert pathologists, and predict whether additional slides should be ordered, and what should the additional ordered slides include (e.g., a type of a specific staining).

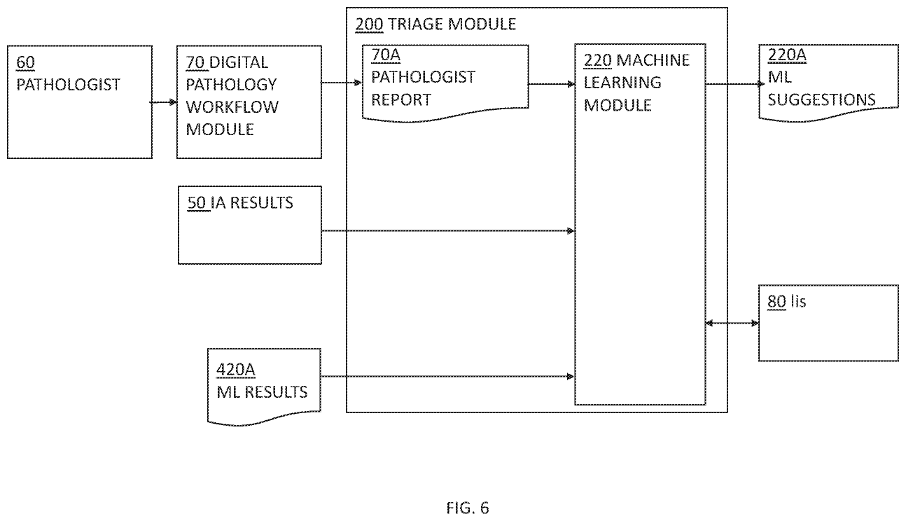

Reference is now made to FIG. 6, which is a block diagram, depicting a triage module 200, which may be included within an embodiment of a system for personalization and optimization of digital pathology analysis, according to some embodiments.

According to some embodiments, at least one extracted feature or result 50 of a first slide 30 associated with a first pathological case may be a first clinical feature 50A, indicative of a first medical condition, and at least one extracted feature or result 50 of a second slide 30 may be a second clinical feature 50A, associated with a second pathological case, indicative of a second medical condition.

Triage module 200 may include an ML module 220, configured to produce a suggestion 220A such as a personalized prioritization suggestion 220A, relating to an examination priority of the pathological case. Prioritization suggestion 220A may, for example, be utilized by one or more pathology experts in the pathology institute, to assign a priority to the examination of the first and second pathological cases and address them according to their respective indications of medical priority or significance, and/or according to a priority or order in which the specific pathologist may prefer.

ML module 220 may include a model (e.g. a supervised model, as known in the art) and may be trained (e.g. by a feedback of at least one pathological report 70A from a pathologist 60) to prioritize cases according to a plurality of factors, including for example: at least one clinical feature or result 50 (e.g., whether a sample is suspected as having a malignant diagnosis 50C, whether a sample includes secondary features 50F that are typically borderline or otherwise difficult to diagnose, and the like); clinical parameters included in the LIS (e.g., medical background of the patient, present condition of the patient, a treating physician's notes, a radiology report, previous test results, results of previous biopsies and the likes); and the pathologist's preferences (e.g., historical selection or order of viewed cases).

According to some embodiments, Triage module 200 may further be configured to produce a suggestion 220A such as a personalized assignment suggestion 220A, relating to assignment of a pathological case to at least one pathologist, according to one or more factors, including for example: at least one clinical feature or result 50; the examination priority (e.g., of a slide, of a pathological case, etc.) as elaborated above, a pathologist's sub-specialty (as may be included in the LIS database 80); efficiency (e.g. time consumption) of each pathologist's diagnosis of specific types of cases (as may be included in the LIS database); a probability of error of each pathologist in the diagnosis of specific types of cases (e.g. 420A, as explained herein); and an availability of one or more pathologists, or the current workload in the pathology institute (e.g., which pathologists are working and how many cases each one is working on, as may be included in the LIS 80 database).

In some embodiments, the assignment of pathological cases in the pathology lab or institute may be implemented as a task list, associating a case to a specific pathologist, and may be stored on a database (e.g., element 6 of FIG. 2) of the institute. Personalized assignment suggestion 220A may, for example, be implemented as an electronic message containing an association of a task (e.g., a serial number thereof) to a pathologist. The message may be sent to a processor (e.g., element 2 of FIG. 2), that may consequently update that task list, to assign the case to a pathologist accordingly.

In some embodiments, ML module 220 may receive (e.g., from the task list stored on database 6) data associated with previous and/or manual assignment of pathological cases to specific pathology experts. ML 220 may be configured to automatically learn that certain types of cases, (e.g. cases that are associated with specific clinical features 50A, such as those containing a specific type of cells), are typically assigned to a specific pathologist in the pathology institute for diagnosis or for second opinion, and may be configured to produce suggestions 220A containing assignment of future cases to specific pathologists according to this learning.

In some embodiments, ML module 220 may produce suggestions 220A such as personalized prioritization suggestions 220A that may include an examination priority of specific pathologists in examination of specific cases, slides and/or locations within slides.

Suggestion 220A may, for example, be manifested or implemented as an electronic message to a processor 2, that may be configured to prioritize specific cases and assign them to specific pathologists (e.g. in a task list stored on database 6), thus saving time and workload of other pathologists in the pathology institute.

Reference is now made to FIG. 7, which is a block diagram, depicting a decision support module 300, which may be included within an embodiment of a system for personalization and optimization of digital pathology analysis, according to some embodiments.

As known in the art, commercially available decision support tools may be used by a pathologist during the analysis or examination of a slide. For example, a decision support tool may present or highlight (e.g. on DPW 70) at least one ROI 50B that may include one or more primary clinical features 50A and may require the pathologist's careful attention. In another example, a decision support tool may compute and display various metrics relating to clinical features the scanned sample, including for example: a size of a suspected tumor, IHC quantification, mitosis counting, etc.

According to some embodiments of the invention, decision support module 300 may include: a workflow features extraction module 310, configured to extract at least one viewing property preference of a user (e.g. a pathology expert) on DPW 70; and an ML module 320, configured to learn the specific user's method of viewing or analyzing the scanned slide, to produce a suggestion 320A such as a personalized viewing suggestion 320A that relates to at least one viewing property. Personalized viewing suggestion 320A may be personalized according to the user's identity and according to at least one of: an IA result 50 associated with the currently examined case (e.g. a clinical feature 50A, an ROI 50B etc.) and the at least one extracted viewing property.

For example, workflow features extraction module 310 may extract at least one viewing property preference, including for example: an order of viewing of each digitally scanned slide of a plurality of slides (e.g., analyze the slides according to their original order, or start with slides that include specific features (e.g., 50A, 50F)); a panning pattern of the presented digital scan by the user (e.g. analyzing the slide from a first end to a second, or starting the scan at areas that include suspicious features (e.g., 50A, 50F) and moving to other, less conspicuous regions); a pattern of magnification of the presented digital scan by the user (e.g., using low magnification for the entire slide and then zooming in at a specific ROIs, or zooming in and out while passing through the ROI); and a selection of at least one of: brightness, contrast, color and gamma correction, and image filters (e.g. high-pass filters) applied by the user.

ML module 320 may include a model (e.g. a supervised model or a non-supervised model, as known in the art) that may be trained according to the at least one IA result 50, and according to at least one extracted viewing property preference, to predict at least one viewing property that may be preferred by a specific user when presented with a new case.

The prediction of the preferred viewing property may be included in suggestion 320A (e.g., a personalized viewing suggestion) and may be propagated (e.g., as an electronic message) to the HMI (e.g. DPW 70). DPW 70 may use suggestion 320A to personalize the viewing of pathological cases, according to individual pathologists' preferences, and according to the currently examined case on the DPW 70 human-machine interface.

As elaborated herein, workflow features extraction module 310 may be configured to extract one or more personal viewing property preferences, pertaining to a specific pathologist. According to some embodiments of the invention, workflow features extraction module 310 may be adapted to produce an “examination story” of a pathologist's examination of a specific pathology case and/or slide, and ML module 320 may be adapted to produce a personalized viewing suggestion 320A based on the examination story.

For example, ML module 320 may determine that a specific pathologist may prefer to perform the examination using a specific viewing pattern (e.g., focusing on areas that include suspicious features (e.g., 50A, 50F) and then moving to other, less conspicuous regions). In a condition in which a pathologist may have started an examination of a scanned pathology slide but needed to pause the examination before its termination (e.g., before producing a pathology report 70A), ML module 320 may produce a personalized viewing suggestion 320A based on the examination story and on the user's preference, to assist the pathologist in resuming the examination. Pertaining to the same example, ML module 320 may produce a personalized viewing suggestion 320A that may adapt DPW 70 to present a region that is predicted to be next in the specific pathologist's preferred order.

As known in the art, a pathologist's workflow normally includes preparation of a pathology report 70A on the analyzed tissue. The report may include observations and conclusions made by the pathologist regarding, for example: clinical findings or features of the scanned slide, diagnosis of the findings, grading and staging of oncological finding, etc. Preparation of a comprehensive pathology report 70A may be very time consuming and may impede the efficiency of the workflow of a pathology institute. According to some embodiments, decision support module 300 may be configured to learn (e.g. by a machine learning algorithm) how specific pathologists prefer to draft their pathological report and what to include in it and produce a report template based on this learning.

For example, workflow features extraction module 310 may be configured to extract at least one feature associated with a pathological report 70A of a user, associated with at least one first digital scan, including for example: features relating to a content of report 70A (e.g., detection of clinical features and associated ROIs, diagnosis of the detected features, etc.); features relating to a format of the pathology report (e.g., ordering of observations in the report, prioritization of detected clinical features, etc.); features relating to appearance of the scanned slides (e.g. a quality of the slide, a request for additional information, a request for an additional slide, a request for an amendment of a scanned slide, etc.); features relating to clinical information that may be included in the LIS (e.g. a treating physician's notes, results of previous tests and biopsies, etc.); and features relating to the workflow in the pathology laboratory or institute (e.g. whether the examined slide was viewed by another pathologist, the workload of the examining pathologist, etc.).

ML module 320 may include a model (e.g. a supervised model or a non-supervised model, as known in the art) that may be trained according to at least one IA result 50 (e.g., a primary clinical feature 50A, a secondary clinical feature 50F, etc.) associated with the at least one first digital scan, and according to at least one feature of the associated pathology report 70A extracted by feature extraction module 310.

ML module 320 may predict, according to its training, at least one element of a pathological report (e.g. content of finding relating to clinical features) that may be preferred by a specific user when presented with a new case.

The predicted at least one element of a pathological report may be included in a report template 320B, that may be used by a pathologist 60 to draft their report in a timewise efficient manner, that may be personalized according to the specific identity of the examining pathologist and optimized according to features (e.g. IA results 50) of the currently examined case and type of examined tissue.

For example, ML module 320 may receive at least one second IA result 50 associated with a second digital scan of a second clinical case. The second IA result 50 may include, for example, a second clinical feature 50A associated with a second ROI 50B. ML module 320 may produce a suggestion 320B, such as a personalized pathological report suggestion 320B, that may include a report template for a second pathological report according to the user's identity, and according to at least one of: the at least one second IA result 50 (e.g., ROI, clinical feature, etc.) and the at least one first pathological report. Personalized pathological report suggestion 320B may be implemented, for example, as a table that may include elements of format (e.g., an order of pathological findings) and/or content (e.g., an essence or characteristics of pathological findings) of the second report, and may be sent (e.g., as an electronic message) to a UI (e.g., to DPW module 70) to present and/or produce the second pathological report.

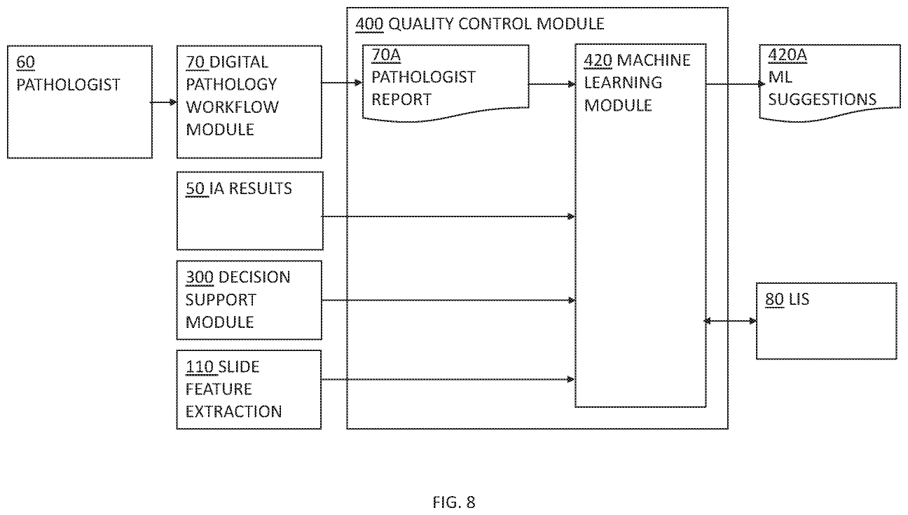

In some embodiments, ML module 320 may produce suggestion (e.g., personalized pathological report suggestion) 320B according to at least one first pathological report 70A of the same pathologist. In alternate embodiments, ML module 320 may produce suggestion 320B according to previous pathological reports of a plurality of pathologists. In some embodiments, data relating to at least one extracted viewing property (e.g. slide coverage and a pattern of magnification by an examining pathologist) and/or at least one ROI 50B may be propagated to a Quality Control (QC) module (e.g. element 400 of FIG. 8), as explained herein. QC module 400 may utilize the propagated data to ascertain whether: (a) a user has properly examined the slide in real time; and (b) a diagnostic error may be explained post-factum (e.g., in the case that a small malignant area may have been missed by the pathologist).

As known in the art, pathology institutes normally implement QC processes to assess and ensure the quality of different aspects of their work. For example, some institutes may arbitrarily select a fraction of the diagnosed cases (e.g., 10%), and send them for a second analysis by another pathologist. Automated QC tools may improve such QC procedures by providing a much larger coverage (e.g., checking all cases instead of just 10%) and by reducing the workload (e.g., a QC tool can point the reviewer to the specific location that looks suspicious, instead of having the reviewer analyze the entire case).

Reference is now made to FIG. 8, which is a block diagram, depicting an automated quality control (QC) module 400, which may be included within an embodiment of a system for personalization and optimization of digital pathology analysis, according to some embodiments. QC module 400 may employ an ML module 420, configured to customize the process of quality assurance and control to specific characteristics and requirements of a pathology institute and pathologists employed therein.

According to some embodiments, ML module 420 may include an ML model (e.g. a supervised ML model or an unsupervised ML model, as known in the art), that may be trained according to a plurality of factors, as explained herein. ML module 420 may produce at least one suggestion 420A such as a personalized second examination suggestion 420A, based on the training of the ML model. The suggestion may relate to a probability of an error that may have been done during an examination, by a pathologist, of at least one scanned slide, and/or to a required additional examination, by a pathologist, of the at least one scanned slide.

According to some embodiments, a user may receive personalized second examination suggestion 420A (e.g. via DPW 70), and may direct the suggestion to a human pathologist, to perform a second reading of the at least one scanned slide. In alternate embodiments, the suggestion 420A may be handled by triage module 200, as depicted in FIG. 6, by assigning the second reading to a pathologist as explained above, in relation to FIG. 6.

QC module 400 may receive from a pathologist (e.g. via DPW 70) at least one indication of an error that may have been made on a first reading of a scanned slide and detected on a second reading of that slide. The indication may include, for example: a detection of a clinical feature, an ROI related to the detected clinical feature, a diagnosis associated with the clinical feature, etc. In some embodiments, the at least one indication of an error may be used by QC module 400 as a supervised feedback for training the model of ML module 420, as known in the art.

According to some embodiments, ML module 420 may receive at least one IA result 50 associated with at least one scanned slide (e.g. a clinical feature associated with an ROI of the slide, a clinical feature associated the entire slide, etc.), and at least one element of a pathological report 70A (e.g. a diagnosis) produced by a user in relation to the at least one scanned slide.

ML module 420 may be trained to prospectively analyze the at least one element of the pathological report, to locate or determine one or more discrepancies between the pathological report and the at least one IA result 50 (e.g. a mismatch between a clinical feature 50A of IA results 50 and a diagnosis within report 70A) and produce a suggestion (e.g., a personalized second examination suggestion) 420A, relating to a required additional examination of the at least one scanned slide.

For example, ML module 420 may detect a condition in which a pathologist may have reported a pathological case as benign in report 70A, whereas at least one IA result 50 associated with the same pathological case may include a clinical feature associated with a high probability of malignancy. In another example, ML module 420 may detect a condition in which a pathologist may have reported a low grade or stage of malignancy in report 70A, whereas at least one IA result 50 may include a higher grade 50D or stage 50E.

In some embodiments, ML module 420 may be trained to predict a probability of malignancy according to at least one IA result 50 and according to historical medical information (e.g., previous pathological results, medical test results, etc.) from LIS module 80. ML module 420 may detect a condition of discrepancy between a pathologist's pathological report 70A and the predicted probability of malignancy. For example, ML module 420 may detect a condition in which a pathologist may have reported a pathological case as benign in report 70A, whereas the predicted probability of malignancy (according to at least one IA result 50 and according to historical medical information) may have been above a predefined threshold.

According to some embodiments, ML module 420 may be trained to retrospectively analyze a plurality of misdiagnosed cases, to detect at least one of: a type of a diagnosis error (e.g. mistaking a specific malignant feature to be a specific benign feature); a risk factor that may be related to the diagnosis error, as elaborated below; and a probability of occurrence of a diagnosis error during analysis of a new scanned slide, in view of the at least one detected risk factor.

QC module 400 may be trained to fine-tune suggestion 420A, so that it would be more sensitive to the types of errors that are more frequent in the institute (e.g., one pathology institute may have more diagnostic errors in skin biopsies, whereas another may have more grading errors in prostate biopsies).

For example, ML module 420 may include at least one classification model of the historical data, that may be trained according to a plurality of historical diagnosis cases to associate each class with a probability of error. ML module 420 may predict, according to the training, a probability of error for new pathological cases, beyond the plurality of historic cases, and produce a suggestion (e.g., a personalized second examination suggestion) 420A, relating to a required additional examination of the new pathological case according to the predicted probability of error.

In some embodiments, QC module 400 may receive a plurality of features and parameters to detect risk factors that may erroneously affect the diagnosis of a pathologist.

For example, QC module 400 may receive, from IA module 40 a plurality of slide clinical features or results 50, including for example: a tissue type, a type of cells, a morphological feature, a diagnosis, a secondary feature, etc. as explained above, in relation to FIG. 3.

In another example, QC module 400 may receive from slide feature extraction module one or more appearance feature relating to at least one quality of a slide, as explained above in relation to FIG. 5. Such appearance features may include, for example: stain colors, scan quality, slice thickness, tissue folds and tears, etc.

In another example, QC module 400 may receive slide related data from LIS 80, including the number of ordered slides and their type (e.g., IHC stains that were ordered for the case).

In another example, QC module 400 may receive clinical data from LIS 80, including, for example: a patient's medical condition and history, results of lab tests, notes by the patient's physician, diagnosis of previous biopsies, radiology report, etc. For example, a prostate biopsy diagnosed as benign for a patient with very high PSA (prostate-specific antigen) levels and a previous prostate biopsy diagnosed as adenocarcinoma, is a-priori more likely to be wrong than a biopsy with similar pathological features, but from a patient with low PSA and no history of adenocarcinoma.

In another example, QC module 400 may receive from LIS 80 information related to the process of diagnosis, including for example: historic data associated with a plurality of historic diagnoses of pathological cases. For example, the historic data may include: parameters of the diagnosis process such as a time of day in which the diagnosis took place (e.g., some pathologists might be more error prone in the late afternoon hours than during the morning), time spent on analyzing the case, etc.; clinical parameters (e.g., previous medical records and/or diagnosis) associated with the respective patient; parameters of previous errors in a diagnosis process (e.g., misdiagnosis of a condition, a grade and/or a stage of a cancerous tumor), and the like.

In another example, QC module 400 may receive from decision support module 300 additional information related to the process of diagnosis, including for example: tissue coverage during the diagnosis, panning and magnification patterns (e.g., whether a pathologist has skipped some parts of the tissue or examined them only at low magnification level), etc.

In some embodiments, QC module 400 may receive from at least one pathology report findings that might be contradicting or may raise suspicion of an error. For example, a pathology report may include a diagnosis of a sample as benign, whereas clinical information of the patient from LIS 80 (e.g., a previous radiology report, a previous biopsy, a previous laboratory tests result, etc.) may indicate a high probability of malignancy.

According to some embodiments, each pathologist may be associated with a specific error profile, that may include, for example at least one of: the pathologist's training, specialty and experience; the pathologist's capabilities (e.g. throughput and/or latency of diagnosis per specific types of clinical cases); and a probability for error of the pathologist per each type of clinical case (e.g., one pathologist may perform better than another when analyzing a specific type of clinical case), and in view of each risk factor (e.g., one pathologist may perform better than another when the task is done late during the work-day).

QC module continuously update the error profile of each pathologist and may propagate at least one element of the profile to the triage module, to assist in selecting a specific pathologist to examine a specific clinical case, as discussed above, in relation to FIG. 6.

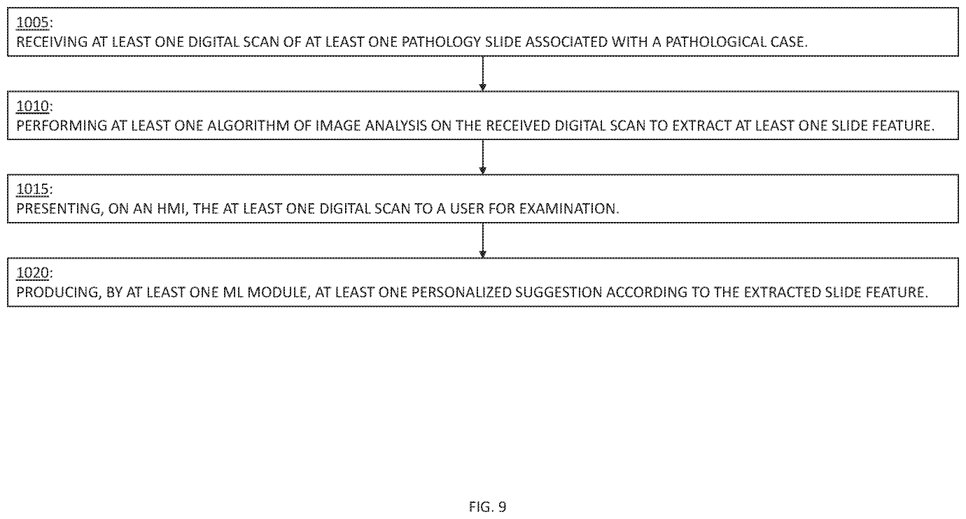

Reference is now made to FIG. 9, which is a flow diagram, depicting a method for personalization and/or optimization of digital pathology analysis by at least one controller and/or processor (e.g., element 2 of FIG. 2), according to some embodiments.

As shown in step 1005, the at least one controller and/or processor 2 may receive at least one digital scan of at least one pathology slide (e.g., element 30 of FIG. 3) associated with a pathological case.

As shown in step 1010, the at least one processor 2 may perform at least one algorithm of image analysis on the received digital scan to extract at least one slide feature. For example, processor 2 may utilize an IA application or module (e.g., element 40 of FIG. 3) to extract at least one slide feature that is a clinical feature (e.g., elements 50A through 50G of FIG. 3) Additionally, or alternatively, processor 2 may utilize a feature extraction module (e.g., element 110 of FIG. 5) extract at least one slide feature that is an appearance feature, as elaborated herein.

As shown in step 1015, the at least one processor 2 may present, on an HMI (e.g., such as DPW module 70 of FIG. 4, output element or device 8 of FIG. 2 and/or input element or device 7 of FIG. 2) the at least one digital scan to a user for examination.

As shown in step 1020, the at least one processor 2 may produce, (e.g., by at least one ML module such as elements 100, 200, 300 and/or 400 of FIG. 4), at least one personalized suggestion (e.g., elements 120A, 220A, 320A and/or 420A) according to the extracted slide feature. Additionally, or alternatively, the at least one processor 2 may produce the at least one personalized suggestion (e.g., elements 120A, 220A, 320A and/or 420A) according to the extracted slide feature and/or according to an identification of a user (e.g., a pathology expert). Additionally, or alternatively, the at least one processor 2 may produce the at least one personalized suggestion (e.g., elements 120A, 220A, 320A and/or 420A) according to the extracted slide feature, according to an identification of the user, and/or according to a pathology report (e.g., element 70A of FIG. 5) and/or a pathology request (e.g., element 70B of FIG. 5) of the pathologist.

Embodiments of the invention provide a practical application for producing pathology reports for digitally scanned pathology slides.

Embodiments of the invention may include an improvement over currently available methods and systems for computed aided diagnosis, by providing personalization of the diagnosis process (e.g., according to specific pathologists' expertise and/or preferences), as elaborated herein.

Furthermore, embodiments of the invention may include an improvement over currently available methods and systems for computed aided diagnosis, by optimizing the diagnosis workflow according to, for example: the types of examined slides, the expertise and preferences of each pathologist, policy of diagnosis in each pathology institute etc., as elaborated herein.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A system for personalization of digital pathology analysis, the system comprising:
an image analysis based computational diagnostics module, configured to receive at least one digital scan of at least one pathology slide associated with a pathological case and perform at least one algorithm of image analysis thereon to extract at least one feature of the at least one slide;
a human-machine interface (HMI) module, configured to present the at least one digital scan to a user for examination; and
at least one machine-learning (ML) module, configured to produce at least one personalized suggestion according to the at least one extracted slide feature.

2. The system according to claim 1, wherein the personalized suggestion is selected from a list consisting of: an assignment suggestion, a preparation suggestion, a prioritization suggestion, a viewing suggestion, a pathological report suggestion and a second examination suggestion.

3. The system according to any one of claims 1 and 2, wherein the HMI is further configured to receive from the user a selection of at least one viewing property, and wherein the at least one ML module is configured to produce the at least one personalized suggestion according to an identity of the user and according to at least one of: the extracted slide feature and the selected viewing property.

4. The system according to any one of claims 1-3, wherein the HMI is further configured to produce a pathological report according to the examination, and wherein the at least one ML module is configured to produce the at least one personalized suggestion according to the identity of the user and according to at least one of: the extracted slide feature, the selected viewing property and the pathological report.

5. The system according to any one of claims 1-4, wherein a first extracted feature of a first slide is a first clinical feature, indicative of a first medical condition and wherein a second extracted feature of a second slide is a second clinical feature, indicative of a second medical condition, and wherein the personalized prioritization suggestion comprises assignment of an examination priority to the slides according to their respective indications of medical conditions.

6. The system according to any one of claims 1-5, further comprising a lab information sub-system (LIS), configured to maintain clinical data selected from a list consisting of: clinical parameters associated with a patient; notes of a treating physician; a report of a radiologist; and a historical biopsy report, wherein the at least one ML module is further configured to produce the personalized prioritization suggestion according to the clinical data of the LIS.

7. The system according to any one of claims 1-6, wherein the LIS is configured to further maintain workflow information selected from a list consisting of: a tissue type associated with the slide, a field of specialization of specific users, a number of pathological cases assigned to each user and an availability of each user, and wherein the at least one ML module is further configured to produce the personalized assignment suggestion according to at least one of: the examination priority and the workflow information.

8. The system according to any one of claims 1-7, wherein at least one pathological report comprises at least one personal appearance preference of a user, and wherein at least one extracted feature is an appearance feature associated with a pathology slide, and wherein the at least one ML module is configured to produce the personalized preparation suggestion according to the user's identification and according to at least one of the personal appearance preference and the appearance feature.

9. The system according to any one of claims 1-8, wherein at least one pathological report comprises at least one pre-ordering preference of a user, and wherein at least one extracted feature of a slide is a clinical feature, indicative of a medical condition, and wherein the at least one ML module is configured to produce a personalized preparation suggestion according to the user's identification and according to at least one of the pre-ordering preference and clinical feature.

10. The system according to any one of claims 1-9, wherein the at least one extracted feature comprises a region of interest (ROI) and a respective clinical feature, and wherein the at least one ML module is configured to produce a personalized viewing suggestion according to the user's identity and according to at least one of: the ROI, the at least one clinical feature and at least one viewing property.

11. The system according to any one of claims 1-10 wherein the at least one ML module is configured to: produce an examination story of a pathologist's examination of a specific pathology slide; extract one or more personal viewing property preferences, pertaining to the specific pathologist; and produce a personalized viewing suggestion according to the user's identity and according to the examination story.

12. The system according to any one of claims 1-11, wherein the at least one viewing property is selected from of a list consisting of: an order of viewing of the plurality of digital scans by the user; a panning pattern of the presented digital scan by the user; a pattern of magnification of the presented digital scan by the user; and a selection of at least one of: brightness, contrast, color and gamma correction by the user.

13. The system according to any one of claims 1-12, wherein at least one first pathological report of a user, associated with a first digital scan, comprises a first ROI associated with a clinical feature, and wherein a second digital scan comprises a second ROI associated with the same clinical feature, and wherein the at least one ML module is configured to produce the personalized report suggestion according to the user's identity and according to at least one of: the second ROI, the clinical feature, and the first pathological report.

14. The system according to any one of claims 1-13, wherein the extracted feature is a clinical feature associated with at least one of an ROI of at least one slide and an entire slide, and wherein the at least one ML module is configured to:
analyze the pathological report to determine one or more discrepancies between the pathological report and the extracted clinical feature; and
produce a personalized second examination suggestion based on the determined one or more discrepancies.

15. The system according to any one of claims 1-14, wherein the at least one ML module is configured to:
obtain historical data associated with a plurality of historic diagnoses of pathological cases from an LIS;
produce at least one classification model of the historical data, associating each class with a probability of error;
predict a probability of error for a new pathological case, beyond the plurality of historic cases; and
produce a second examination suggestion according to the predicted probability of error.

16. The system according to any one of claims 1-15, wherein the historical data comprises at least one of:
parameters of the diagnosis process;
a clinical feature of at least one slide associated with the pathological case;
clinical parameters associated with the respective patient; and
parameters of an error in the diagnosis process.

17. A method for personalization of digital pathology analysis by at least one processor, the method comprising:
receiving at least one digital scan of at least one pathology slide associated with a pathological case;
performing at least one algorithm of image analysis on the received digital scan to extract at least one slide feature;
presenting, on an HMI the at least one digital scan to a user for examination; and
producing, by at least one ML module, at least one personalized suggestion according to the extracted slide feature.

18. The method according to claim 17, wherein the personalized suggestion is selected from a list consisting of: an assignment suggestion, a preparation suggestion, a prioritization suggestion, a viewing suggestion, a pathological report suggestion and a second examination suggestion.

19. The method according to any one of claims 17 and 18, further comprising:
receiving, by the HMI, a selection of at least one viewing property; and
producing the at least one personalized suggestion according to an identity of the user and according to at least one of: the extracted slide feature and the selected viewing property.

20. The method according to any one of claims 17-19, further comprising:
producing, by the HMI, a pathological report according to the examination; and
producing the at least one personalized suggestion according to the identity of the user and according to at least one of: the extracted slide feature, the selected viewing property and the pathological report.

21. The method according to any one of claims 17-20, wherein a first extracted feature of a first slide is a first clinical feature, indicative of a first medical condition and wherein a second extracted feature of a second slide is a second clinical feature, indicative of a second medical condition, and wherein the personalized prioritization suggestion comprises assignment of an examination priority to the slides according to their respective indications of medical conditions.

22. The method according to any one of claims 17-21, further comprising:
maintaining clinical data on an LIS; and
producing at least one personalized prioritization suggestion according to the clinical data.

23. The method according to any one of claims 17-22, wherein the clinical data is selected from a list consisting of: clinical parameters associated with a patient; notes of a treating physician; a report of a radiologist; and a historical biopsy report.

24. The method according to any one of claims 17-23, further comprising:
maintaining workflow information on the LIS, selected from a list consisting of: a tissue type associated with the slide, a field of specialization of specific users, a number of pathological cases assigned to each user and an availability of each user; and
producing the personalized assignment suggestion according to at least one of: the examination priority and the workflow information.

25. The method according to any one of claims 17-24, wherein at least one pathological report comprises at least one personal appearance preference of a user, and wherein at least one extracted feature is an appearance feature associated with a pathology slide, and wherein the method further comprises producing the personalized preparation suggestion according to the user's identification and according to at least one of the personal appearance preference and the appearance feature.

26. The method according to any one of claims 17-25, wherein at least one pathological report comprises at least one pre-ordering preference of a user, and wherein at least one extracted feature of a slide is a clinical feature, indicative of a medical condition, and wherein the method further comprises producing the personalized preparation suggestion according to the user's identification and according to at least one of the pre-ordering preference and clinical feature.

27. The method according to any one of claims 17-26, wherein the at least one extracted feature comprises an ROI and a respective clinical feature, and wherein the method further comprises producing the personalized viewing suggestion according to the user's identity and according to at least one of: the ROI, the at least one clinical feature and at least one viewing property.

28. The method according to any one of claims 17-27, further comprising:
producing an examination story of a pathologist's examination of a specific pathology slide;
extracting one or more personal viewing property preferences, pertaining to the specific pathologist; and
producing a personalized viewing suggestion according to the user's identity and according to the examination story.

29. The method according to any one of claims 17-28, wherein the at least one viewing property is selected from of a list consisting of: an order of viewing of the plurality of digital scans by the user; a panning pattern of the presented digital scan by the user; a pattern of magnification of the presented digital scan by the user; and a selection of at least one of: brightness, contrast, color and gamma correction by the user.

30. The method according to any one of claims 17-29, wherein at least one first pathological report of a user, associated with a first digital scan, comprises a first ROI associated with a clinical feature, and wherein a second digital scan comprises a second ROI associated with the same clinical feature, and wherein the method further comprises producing the personalized report suggestion according to the user's identity and according to at least one of: the second ROI, the clinical feature, and the first pathological report.

31. The method according to any one of claims 17-30, wherein the extracted feature is a clinical feature associated with at least one of an ROI of at least one slide and an entire slide, and wherein the method further comprises:
analyzing the pathological report to determine one or more discrepancies between the pathological report and the extracted clinical feature; and
producing a personalized second examination suggestion based on the determined one or more discrepancies.

32. The method according to any one of claims 17-31, further comprising:
obtaining historical data associated with a plurality of historic diagnoses of pathological cases from an LIS;
producing at least one classification model of the historical data, associating each class with a probability of error;
predicting a probability of error for a new pathological case, beyond the plurality of historic cases; and
producing a second examination suggestion according to the predicted probability of error.

33. The method according to any one of claims 17-32, wherein the historical data comprises at least one of:
parameters of the diagnosis process;
a clinical feature of at least one slide associated with the pathological case;
clinical parameters associated with the respective patient; and
parameters of an error in the diagnosis process.
